(19) United States
(12) Patent Application Publication
(10) Pub. No.: US 2015/0079007 A1
(43) Pub. Date: Mar. 19, 2015

(54) TUNABLE MULTIMODAL NANOPARTICLES AND METHODS OF USE THEREOF

(71) Applicant: Board of Regents of the University of Nebraska, Lincoln, NE (US)

(72) Inventors: Xin-Ming Liu, Omaha, NE (US); Alexander V. Kabanov, Chapel Hill, NC (US); Howard E. Gendelman, Omaha, NE (US)

(73) Assignee: Board of Regents of the University of Nebraska, Lincoln, NE (US)

(21) Appl. No.: 14/394,025

(22) PCT Filed: Apr. 22, 2013
(86) PCT No.: PCT/US2013/037582

§ 371 Date: Oct. 10, 2014

Related U.S. Application Data
(60) Provisional application No. 61/636,029, filed on Apr. 20, 2012.

(51) Int. Cl. A61K 9/50; A61K 49/00; A61K 49/18

(52) U.S. Cl. CPC A61K 9/5031; A61K 49/1857; A61K 49/0002
USPC 424/9.322; 424/9.1

(57) ABSTRACT

Tunable nanoparticles and methods of use thereof are provided. In accordance with the instant invention, tunable nanoparticles comprising a metal nanoparticle core (e.g., a paramagnetic particle (e.g., USPIO) or a quantum dot) and a polymer linked to a metal binding moiety are provided. The polymer of the nanoparticle is bound to the metal nanoparticle core by the metal binding moiety and coats the metal nanoparticle core. In a particular embodiment, the metal binding moiety comprises bisphosphonate, pyrophosphate, or a derivative thereof. In a particular embodiment, the polymer is a hydrophilic polymer, an amphiphilic block copolymer or an ionic block copolymer.

This application claims priority under 35 U.S.C. §119(e) to U.S. Provisional Patent Application No. 61/636,029, filed on Apr. 20, 2012. The foregoing applications are incorporated by reference herein.

This invention was made with government support under Grant No. 1P01 DA028555 awarded by the National Institutes of Health. The government has certain rights in the invention.

### FIELD OF THE INVENTION

The present invention relates generally to nanoparticles and methods of use thereof. The present invention also relates to compositions and methods for the delivery of therapeutic and diagnostic agents to a subject.

### BACKGROUND OF THE INVENTION

Microglial neuroinflammatory response plays a central role in the pathogenesis and progression of Alzheimer's and Parkinson's diseases (McGeer et al. (1995) Brain Res. Brain Res. Rev., 21:195; Glass et al. (2010) Cell 140:918). Control of such neuroinflammatory responses can reduce or modulate the production or release of neurotoxic factors that contribute to neuronal demise and associated neurological conditions that affect clinical benefit. Paradoxically, clinical intervention trials have not shown that long term control of neuroinflammation, either through vaccination or by administration of nonsteroidal anti-inflammatory drugs can alter the disease course (in t'Veld et al. (2001) N. Engl. J. Med., 345:1515).

Pathologically, aggregates amylold beta peptide (Aβ), called senile plaques in AD, and aggregates of the α-synuclein protein called Lewy bodies in PD, activate microglia and induce the production of reactive oxygen species (ROS) and proinflammatory cytokines and chemokines, which further induce Aβ and α-synuclein protein production from neurons and astrocytes. For AD, inflammatory factors also act directly on cholinergic neurons and stimulate astrocytes to amplify proinflammatory signals and induce neurotoxic effects, apoptosis, and necrosis of neurons further activates microglia. For PD, inflammatory factors act directly on dopaminergic neurons of the substantia nigra to induce neurotoxic effects, inflammatory factors also further activate microglia to amplify the inflammatory response, products derived from microglia and astrocytes act in a combinatorial manor to promote nerotoxicity. This glia-induced vicious cycle continues and leads to chronic, sustained and progressive neuroinflammation, which significantly exacerbates the pathogenic processes of AD and PD (Glass et al. (2010) Cell 140:918; Hardy et al. (2002) Science 297:353; Tanzi et al. (2005) Cell 120:545). Clearly, there is a need for better therapeutics and earlier detection methods (Akiyama et al. (2000) Neurobiol. Aging 21:383).

### SUMMARY OF THE INVENTION

In accordance with the instant invention, tunable nanoparticles comprising a metal nanoparticle core (e.g., a paramagnetic particle (e.g., USPIO) or a quantum dot) and a polymer linked to a metal binding moiety are provided. The polymer of the nanoparticle is bound to the metal nanoparticle core by the metal binding moiety and coats the metal nanoparticle core. In a particular embodiment, the metal binding moiety comprises bisphosphonate, pyrophosphate, or a derivative thereof. In a particular embodiment, the polymer is a hydrophilic polymer, an amphiphilic block copolymer or an ionic block copolymer. The nanoparticles of the instant invention may further comprise a therapeutic agent, such as an anti-inflammatory, chemotherapeutic, anti-microbial, or the like. When the therapeutic agent is hydrophobic, the polymer may be an amphiphilic block copolymer such that a hydrophobic block of the amphiphilic block copolymer encapsulates the hydrophobic therapeutic agent and a hydrophilic block coats the surface of the nanoparticle. When the therapeutic agent is charged, the polymer may be an ionic block copolymer wherein the charge of the ionic block copolymer is the opposite of the therapeutic agent such that an ionically charged block of the ionic block copolymer encapsulates the charged therapeutic agent and a hydrophilic block coats the surface of the nanoparticle. In a particular embodiment, at least a portion of the polymers of the nanoparticle are linked to at least one targeting ligand, such as a cancer targeting ligand or a macrophage targeting ligand. In a particular embodiment, the targeting ligand is attached to a hydrophilic segment/portion of the polymer. Compositions comprising at least one nanoparticle of the instant invention and, optionally, at least one pharmaceutically acceptable carrier are also provided.

In accordance with another aspect of the instant invention, methods of treating, inhibiting, and/or preventing a disease or disorder (e.g., a neurodegenerative disease, cancer, inflammatory disease, infectious disease, etc.) in a subject are provided. The methods comprise administering at least nanoparticle of the instant invention to the subject. Methods for monitoring the biodistribution of a therapeutic agent and/or determining the efficacy of a therapy in a subject are also provided.

### BRIEF DESCRIPTIONS OF THE DRAWING

FIG. 1 provides a schematic for the chemical synthesis of alendronate conjugated to a polyethylene oxide polymer.

FIG. 2A provides the stability results (size and polydispersity index (PDI) change) of ultrasmall superparamagnetic iron oxide particles (USPIOs) coated with alendronate-PEG under various pH and salt conditions. FIG. 2B provides transmission electron microscopy (TEM) images of USPIOs coated with alendronate-PEG after storage for 2 weeks at room temperature under various pH and salt conditions. FIG. 2C provides images of USPIOs coated with alendronate-PEG under various pH and salt conditions for either 1 month (top panel) or 2 months (bottom panel), thereby demonstrating the superior stability of the alendronate-PEG coating on USPIO.

FIG. 3A provides the stability results (size and PDI change) of ferumoxytol (Feraheme™) under various pH and salt conditions. FIG. 3B provides transmission electron microscopy (TEM) images of ferumoxytol after storage for 2 weeks at room temperature under various pH and salt conditions. FIG. 3C provides images of ferumoxytol under various pH and salt conditions for either 1 month (top panel) or 2 months (bottom panel).

FIG. 4 provides thermal gravimetric analysis (TGA) profiles of USPIOs coated with various ratios of alendronate-PEG, which demonstrate the tunable and highly efficient coating of alendronate-PEG on USPIO. OA-M: oleic acid coated USPIO; AP-M: alendronate-PEG coated USPIO; AP: alendronate-PEG; AP-M-05, 1, 2, 4, or 10: the weight ratio of alendronate-PEG to oleic acid coated USPIO is 0.5, 1, 2, 4, or 10.

FIG. 5A provides a graph of viability of monocyte-derived macrophages (MDM) after incubation with various concentrations of alendronate-PEG coated USPIOs compared to ferumoxytol, thereby demonstrating the biocompatibility of alendronate-PEG coated USPIOs. FIG. 5B provides images of MDM cells incubated with alendronate-PEG coated USPIOs (right panel) or ferumoxytol (left panel). FIG. 5C provides a graph of the MRI relaxivity studies for alendronate-PEG coated USPIOs (AP-M) and ferumoxytol (Feraheme™).

FIG. 6 provides a schematic of the formation of a nanoparticle comprising block ionomer complexes (BIC) formed by negatively charged PEO-PLD-ALN and positively charged therapeutic agents around a USPIO core.

FIG. 7 provides a schematic for the synthesis of L-PEO-PLD (poly-L-aspartic acid)-ALN.

### DETAILED DESCRIPTION OF THE INVENTION

Alzheimer's disease (AD) affects more than 5 million people in the United States and 37 million worldwide, and is recognized as the most common form of dementia. The prevalence of AD is expected to triple over the next 50 years (Mayeux, R. (2010) N. Engl. J. Med., 362:2194; Rafii et al. (2009) BMC Med., 7:7). Parkinson's disease (PD) is the second most common neurodegenerative disease after AD and is the most common movement disorder. About 2% of the population over the age of 60 is affected (Rajabally et al. (2011) Neurology 77:1947). Although the pathophysiologic mechanism of AD and PD is not fully understood, neuroinflammation plays an important role in the pathogenesis of chronic neurodegenerative disorders in AD and PD patients.

Currently, no therapy is available to cure AD and PD and there is an unmet medical need to develop effective “disease-modifying” therapies for these neurodegenerative disorders. Drug research and development for AD and PD has increased dramatically in recent years and one of the main areas of focus is the development of anti-neuroinflammatory therapeutics (Rafii et al. (2009) BMC Med., 7:7). However, large-scale double-blind placebo-controlled clinical trials have not supported the use of NSAIDs for treating neurodegenerative disorders (in t′ Veld et al. (2001) N. Engl. J. Med., 345:1515; Trepanier et al. (2010) J. Alzheimer's Dis., 21:1089). There is significant interest and efforts in developing more effective and selective agents that prevents and/or ameliorates neuroinflammation (Lin et al. (2012) J. Azheimer's Dis., 29:659).

Imaging plays critical roles in diagnosis, treatment, and confirmation of therapy. Obviously, current anti-neuroinflammation strategies for AD and PD are expected to achieve their maximum efficacy if these putative therapeutic agents are initiated in the early progression stage of AD and PD and delivered to the disease target in a personalized manner based on image-guided drug delivery (Beckmann et al. (2010) J. Neurosci., 31:1023). Hence, better ways to noninvasively detect and treat AD and PD related pathology in preclinical and clinical stages are urgently needed.

Herein, polymers modified with metal binding moieties (e.g., bisphosphonates, pyrophosphonates) were used to develop multimodal nanoparticles (e.g., nanotheranostics). The nanoparticles can be used to carry any therapeutic agent and/or imaging agent for the treatment or diagnosis of any disease. As an example, the instant invention specifically provides nanoparticles for the treatment and/or diagnosis of neuro inflammation, particularly in AD and PD. This system will not only allow the noninvasive real-time assessment of drug pharmacokinetics/biodistribution profiles with clinical efficacy, but also allow the development of diagnostics and therapeutics for personalized treatment (e.g., for AD and PD). While the instant invention describes the use of the nanoparticles in the diagnosis and/or treatment of neurological disorders such as Alzheimer's disease and Parkinson's disease, the nanoparticles may also be used in diagnostic and/or therapeutic applications of any other disease or disorder such as other degenerative diseases, cancer, and microbial infections.

Superparamagnetic iron oxide particles (SPIOs (e.g., ultrasmall superparamagnetic iron oxide particles (USPIOs)) are preferred particles due to their high relaxation values and clinically acceptable biocompatibility (Mahmoudi et al. (2011) Adv. Drug Deliv. Rev., 63:24). SPIOs have been widely used for in vivo biomedical applications including MRI, image-guided drug delivery and hyperthermia therapy (Kievit et al. (2011) Accounts Chem. Res., 44:853; Kumar et al. (2011) Adv. Drug Deliv. Rev., 63:789; Veiseh et al. (2010) Adv. Drug Deliv. Rev., 62:284). For imaging, SPIO of >50 nm are rapidly cleared from the blood circulation by cells of the mononuclear phagocytic system (MPS) with a short plasma half-life. Accordingly, they are less optimal for vascular or central nervous system (CNS) imaging (Varallyay et al. (2002) AJNR 23:510). USPIOs of <50 nm are not immediately recognized by MPS and have a longer blood half-life and, therefore, are widely studied for CNS MRI (Manninger et al. (2005) AJNR 26:2290; Neuwelt et al. (2007) Neurosurgery 60:601). MRI with USPIOs depends on their intracellular uptake and retention by phagocytic white blood cells such as monocytes, macrophages, reactive astrocytes, and activated microglia (Weinstein et al. (2010) J. Cerebral Blood Flow Metabol., 30:15). These phagocytic cells dominate neuroinflammation. Therefore, one application of USPIOs of the instant invention is for in vivo cell-specific MRI neuroimaging to noninvasively assess neuroinflammatory processes and evaluate therapeutic efficacy (Weinstein et al. (2010) J. Cerebral Blood Flow Metabol., 30:15; Hamilton et al. (2011) Am. J. Roentgenol. 197:981). For in vivo imaging, the coating of USPIOs is required to be very stable and rigid to avoid coating dissociation. Indeed, bare USPIO exposure to cellular components and organelles subsequently results in cellular toxicity (Tassa et al. (2011) Accounts Chem. Res., 44:842).

Several SPIOs coated with different polymers and ligands have been developed for neuroinflammation detection in various neurodegenerative diseases (Yang et al. (2011) Neuroimage 55:1600; Thorek et al. (2011) Mol. Imaging. 10:206). For example, due to its stability and biocompatibility, ferumoxylol (Feraheme™, AMAG Pharmaceuticals) coated with semi-synthetic dextran may be used for CNS MRI. Currently, various complimentary combinations of imaging modalities and targeting strategies have been developed for optimized disease detection, including those for AD and PD (Yang et al. (2011) Neuroimage 55:1600; Yang et al. (2011) Biomaterials 32:4151; Das et al. (2009) Small 5:2883; Lamanna et al. (2011) Biomaterials 32:8562). However, the non-controllable chemical modification and ligand conjugation complicates the coating structure of USPIOs with non-reproducible results. In contrast, the nanoparticles of the instant invention provide numerous advantages over prior SPIO including, without limitation: 1) a stable, tunable, multimodal platform; 2) the ability to combine diagnostic and therapeutic measures in the same particle; 3) the results provided herein show that ALN-PEO coating of SPIOs are very rigid and stable in wide ranges of pH and salt solutions, thereby avoiding coating disassociation, bare SPIO exposure, and cellular toxicity in vivo; 4) the amount of ALN-PEO coating on SPIOs can be tuned in a wide range, which allows for the incorporation of one or more targeting ligands, one or more imaging agents, and/or one or more therapeutic agents into a single nanoparticle; and 5) SPIOs of the instant invention also serve as stable anchors for block ionomer complexes (BIC) formed by negatively charged PEO-PLD-ALN and positively charged therapeutic agents, thereby avoiding in vivo BIC disintegration and subsequent fast drug release due to pH and salt that may exist in non-cross-linked PEO-PLD or PEO-PEI based BICs.

As explained herein, the instant invention encompasses nanoparticles for the delivery of compounds to a cell. In a particular embodiment, the nanoparticle is for the delivery of a therapeutic agent to a subject. In a particular embodiment, the nanoparticle of the instant invention is up to 1 μm in diameter, particularly about 5 nm to about 500 nm, about 5 to about 200 nm, or about 10 to about 50 nm in diameter. In a particular embodiment, the nanoparticles have a PDI of less than about 0.25. The nanoparticles of the instant invention may comprise at least one metal particle and at least one coating compound (e.g., encapsulating compound) modified by at least one metal binding moiety. The nanoparticles may further comprise at least one therapeutic agent, at least one imaging agent, and/or at least one targeting ligand. The components of the nanoparticle, along with other optional components, are described in more detail hereinbelow.

### I. METAL PARTICLES

The nanoparticles of the instant invention comprise at least one metal nanoparticle. In a particular embodiment, the metal nanoparticle is paramagnetic or superparamagnetic. In a particular embodiment, the metal nanoparticle has a diameter less than about 100 nm, less than about 50 nm, less than about 25 nm, or less than about 10 nm. The metal of the nanoparticles may be, without limitation, iron, gold, cobalt, nickel, gadolinium, dysprosium, praseodymium, europium, manganese, protactinium, chromium, copper, titanium, or vanadium. In a particular embodiment, the metal is iron, gold, cobalt, nickel, gadolinium, or dysprosium. Examples of paramagnetic ions include, without limitation, Au(II), Gd(III), Eu(III), Dy(III), Pr(III), Pa(IV), Mn(II), Cr(III), Co(III), Fe(III), Cu(II), Ni(II), Ti(III), and V(IV). In a particular embodiment, then metal particle comprises iron oxide (e.g., magnetite). The metal particle of the instant invention may also be a quantum dot (e.g., a semiconductor nanocrystal with a diameter from about 2 nm and about 50 nm). The metal particles may be at least partly modified or coated (although not completely coated) prior to attachment of coating compound. For example, the metal particle may be hydrophobically modified (e.g., with oleic acid), particularly when the bioactive agent (e.g., therapeutic agent) is hydrophobic.

In a particular embodiment, the iron oxide particle is a superparamagnetic iron oxide particle (SPIO), particularly an ultrasmall superparamagnetic iron oxide particle (USPIO) or quantum dot. As explained hereinabove, SPIOs and USPIOs are desirable particles due to their high relaxation values, clinically acceptable biocompatibility, and utility for in vivo biomedical applications including MRI (Mahmoudi et al. (2011) Adv. Drug Deliv. Rev., 63:24; Kievit et al. (2011) Accounts Chem. Res., 44:853; Kumar et al. (2011) Adv. Drug Deliv. Rev., 63:789; Veiseh et al. (2010) Adv. Drug Deliv. Rev., 62:284).

### II. METAL BINDING MOIETY

The nanoparticles of the instant invention also comprise a coating compound (e.g., a polymer) conjugated to a metal binding moiety. The metal binding moiety anchors the coating compound to the surface of the metal particle. Metal binding moieties are those compounds which preferentially accumulate in/on metal surfaces rather than other surfaces or any surrounding cells, organs or tissues. Metal binding moieties of the instant invention include, without limitation: bisphosphonates (e.g., alendronate, pamidronate, neridronate, etidronate, ibandronate, zoledronate, risendronate), pyrophosphates, and derivatives thereof. In a particular embodiment, the metal binding moiety is alendronate.

The metal binding moiety may be linked directly to the coating compound or via a linker. Generally, the linker is a chemical moiety comprising a covalent bond or a chain of atoms that covalently attaches the metal binding moiety to the coating compound. The linker can be linked to any synthetically feasible position of the metal binding moiety and the coating compound. Exemplary linkers may comprise at least one optionally substituted; saturated or unsaturated; linear, branched or cyclic alkyl group or an optionally substituted aryl group (e.g., the linker may comprise from 1 to about 100 atoms). The linker may also be a polypeptide (e.g., from about 1 to about 10 amino acids, particularly about 1 to about 5). The linker may be non-degradable and may be a covalent bond or any other chemical structure which cannot be substantially cleaved or cleaved at all under physiological environments or conditions.

### III. COATING COMPOUND

As stated above, the nanoparticles of the instant invention also comprise a coating compound conjugated to a metal binding moiety. Bare metal particles can have toxic side effects in vivo. Accordingly, the coating compound of the nanoparticles of the instant invention serves, in part, to mask the core metal particle. In a particular embodiment, the coating compound is a polymer. For example, the coating compound may be a hydrophilic polymer, an amphiphilic copolymer, a block copolymer, an ionic block copolymer, or an amphiphilic block copolymer. The polymers may be natural polymers, synthetic polymers or semi-synthetic polymers. The polymers of the instant invention may have capping termini.

In a particular embodiment, the coating compound is a hydrophilic polymer or an amphiphilic copolymer, particularly an amphiphilic block copolymer. The hydrophilic polymer may be a homopolymers, copolymer, or block copolymer. The hydrophilic polymer is preferably biocompatible. Examples of biocompatible hydrophilic polymers include, without limitation: polyetherglycols, polyethylene glycol (PEG), methoxy-poly(ethylene glycol), proteins, gelatin, albumin, peptides, DNA, RNA, polysaccharides, dextran, polyvinyl alcohol, polyvinyl pyrrolidone, polyvinyltriazole, N-oxide of polyvinylpyridine, N-(2-hydroxypropyl)methacrylamide (HPMA), polyortho esters, polyglycerols, polyacrylamide, polyoxazolines (e.g., methyl or ethyl poly(2-oxazolines)), polyacroylmorpholine, and copolymers or derivatives thereof.

The amphiphilic compound may be, for example, a surfactant or a lipid (e.g., a phosholipid), optionally linked to a hydrophilic compound or polymer as described herein (e.g., PEO, polysaccharide, particularly to the head group). Amphiphilic block copolymers may comprise two, three, four, five, or more blocks (segments). For example, the amphiphilic block copolymer may be of the general formula A-B, B-A, A-B-A, B-A-B, A-B-A-B-A, or B-A-B-A-B, wherein A represents a hydrophilic block and B represents a hydrophobic block. The amphiphilic block copolymers may be in a linear formation or a branched, hyper-branched, dendrimer, graft, or star formation (e.g., A(B)ₙ, (AB)ₙ, AₙBₘ, starblocks, etc.). The blocks of the amphiphilic block copolymers can be of variable length. In a particular embodiment, the blocks of the amphiphilic block copolymer independently comprise from about 2 to about 1000 repeating units, particularly from about 5 to about 200 or about 5 to about 150 repeating units.

The blocks of the amphiphilic block copolymer may comprise a single repeating unit. Alternatively, the blocks may comprise combinations of different hydrophilic or hydrophobic units. Hydrophilic blocks may even comprise hydrophobic units so long as the character of the block is still hydrophilic (and vice versa). For example, to maintain the hydrophilic character of the block, the hydrophilic repeating unit would predominate over any hydrophobic unit.

In a particular embodiment, the hydrophilic segments are represented by polymers with aqueous solubility more that about 1% wt. at 37° C., while hydrophobic segments are represented by polymers with aqueous solubility less than about 1% wt. at 37° C. In a particular embodiment, polymers that at 1% solution in bi-distilled water have a cloud point above about 37° C., particularly above about 40° C., represent the hydrophilic segments. In a particular embodiment, polymers that at 1% solution in bi-distilled water have a cloud point below about 37° C., particularly below about 34° C., represent the hydrophobic segments.

The amphiphilic compound is preferably biocompatible. Examples of biocompatible amphiphilic copolymers are known in the art, including, for example, those described in Gaucher et al. (J. Control Rel. (2005) 109:169-188). Examples of amphiphilic block copolymers include, without limitation: poly(2-oxazoline) amphiphilic block copolymers, polyethylene glycol-polylactic acid (PEG-PLA), PEG-PLA-PEG, polyethylene glycol-polyanhydride, polyethylene glycol-poly(lactic-co-glycolic acid) (PEG-PLGA), polyethylene glycol-polycaprolactone (PEG-PCL), polyethylene glycol-polyaspartate (PEG-PAsp), polyethylene glycol-poly(glutamic acid) (PEG-PGlu), polyethylene glycol-poly(acrylic acid) (PEG-PAA), polyethylene glycol-poly(methacrylic acid) (PEG-PMA), polyethylene glycol-poly(ethyleneimine) (PEG-PEI), polyethylene glycol-poly(L-lysine) (PEG-PLys), polyethylene glycol-poly(2-(N,N-dimethylamino)ethyl methacrylate) (PEG-PDMAEMA), polyethylene glycol-chitosan, and derivatives thereof.

Examples of hydrophilic block(s) include, without limitation, the hydrophilic polymers described above, particularly: polyetherglycols, dextran, gelatin, albumin, poly(ethylene oxide), methoxy-poly(ethylene glycol), copolymers of ethylene oxide and propylene oxide, polysaccharides, polyvinyl alcohol, polyvinyl pyrrolidone, polyvinyltriazole, N-oxide of polyvinylpyridine, N-(2-hydroxypropyl) methacrylamide (HPMA), polyortho esters, polyglycerols, polyacrylamide, polyoxazolines (e.g., methyl or ethyl poly(2-oxazolines)), polyacroylmorpholine, and copolymers or derivatives thereof. In a particular embodiment, the hydrophilic block(s) of the amphiphilic block copolymer comprises poly(ethylene oxide) (also known as polyethylene glycol).

In a particular embodiment, the hydrophobic block(s) of the amphiphilic block copolymer comprises polyester, poly(lactic acid), poly(lactic-co-glycolic acid), poly(lactic-co-glycolide), polyanhydride, poly aspartic acid, polyoxazolines (e.g., butyl, propyl, pentyl, nonyl, or phenyl poly(2-oxazolines)), poly glutamic acid, polycaprolactone, poly(propylene oxide), poly(1,2-butylene oxide), poly(n-butylene oxide), poly(ethyleneimine), poly(tetrahydrofurane), ethyl cellulose, polydipyrolle/dicabazole, starch, and/or poly(styrene).

In a particular embodiment, the amphiphilic block copolymer comprises at least one block of poly(oxyethylene) and at least one block of poly(oxypropylene). Polymers comprising at least one block of poly(oxyethylene) and at least one block of poly(oxypropylene) are commercially available under such generic trade names as “lipoloxamers”, “Pluronic®,” “poloxamers,” and “synperonics.” Examples of poloxamers include, without limitation, Pluronic® L31, L35, F38, L42, L43, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R2, 17R4, 17R8, 22R4, 25R1, 25R2, 25R4, 25R5, 25R8, 31R1, 31R2, and 31R4. Pluronic® block copolymers are designated by a letter prefix followed by a two or a three digit number. The letter prefixes (L, P, or F) refer to the physical form of each polymer, (liquid, paste, or flakeable solid). The numeric code defines the structural parameters of the block copolymer. The last digit of this code approximates the weight content of EO block in tens of weight percent (for example, 80% weight if the digit is 8, or 10% weight if the digit is 1). The remaining first one or two digits encode the molecular mass of the central PO block. To decipher the code, one should multiply the corresponding number by 300 to obtain the approximate molecular mass in daltons (Da). Therefore Pluronic® nomenclature provides a convenient approach to estimate the characteristics of the block copolymer in the absence of reference literature. For example, the code ‘F127’ defines the block copolymer, which is a solid, has a PO block of 3600 Da (12×300) and 70% weight of EO. The precise molecular characteristics of each Pluronic® block copolymer can be obtained from the manufacturer.

In a particular embodiment, the coating compound is a block copolymer comprising at least one ionically charged polymeric block and at least one non-ionically charged polymeric block (e.g., hydrophilic block). In a particular embodiment, the block copolymer has the structure A-B or B-A. The block copolymer may also comprise more than 2 blocks (e.g., 3, 4, 5, or more). For example, the block copolymer may have the structure A-B-A, wherein B is an ionically charged polymeric block. In a particular embodiment, the blocks/segments of the block copolymer independently comprise about 2 to about 1000 repeating units, particularly from about 5 to about 200 or about 5 to about 150 repeating units.

Examples of hydrophilic blocks are provided hereinabove. The ionically charged polymeric block may be cationic or anionic. The ionically charged polymeric block may also be hydrophobic. The anionically charged polymeric segment may be selected from, without limitation, polymethylacrylic acid and its salts, polyacrylic acid and its salts, copolymers of acrylic acid and its salts, poly(phosphate), polyamino acids (e.g., polyglutamic acid, polyaspartic acid), polymalic acid, polylactic acid, homopolymers or copolymers or salts thereof of aspartic acid, 1,4-phenylenediacrylic acid, ciraconic acid, citraconic anhydride, trans-cinnamic acid, 4-hydroxy-3-methoxy cinnamic acid, p-hydroxy cinnamic acid, trans glutaconic acid, glutamic acid, itaconic acid, linoleic acid, linlenic acid, methacrylic acid, maleic acid, trans-β-hydromuconic acid, trans-trans muconic acid, oleic acid, vinylsulfonic acid, vinyl phosphonic acid, vinyl benzoic acid, and vinyl glycolic acid and the like and carboxylated dextran, sulfonated dextran, heparin and the like.

Examples of polycationic blocks include but are not limited to polymers and copolymers and their salts comprising units deriving from one or several monomers including, without limitation: primary, secondary and tertiary amines, each of which can be partially or completely quaternized forming quaternary ammonium salts. Examples of these monomers include, without limitation, cationic amino acids (e.g., lysine, arginine, histidine), alkyleneimines (e.g., ethyleneimine, propyleneimine, butyleneimine, pentyleneimine, hexyleneimine, and the like), spermine, vinyl monomers (e.g., vinylcaprolactam, vinylpyridine, and the like), acrylates and methacrylates (e.g., N,N-dimethylaminoethyl acrylate, N,N-dimethylaminoethyl methacrylate, N,N-diethylaminoethyl acrylate, N,N-diethylaminoethyl methacrylate, t-butylaminoethyl methacrylate, acryloxyethyltrimethyl ammonium halide, acryloxyethyl-dimethylbenzyl ammonium halide, methacrylamidopropyltrimethyl ammonium halide and the like), allyl monomers (e.g., dimethyl diallyl ammoniam chloride), aliphatic, heterocyclic or aromatic ionenes. In a particular embodiment, the cationic polymeric segment comprises cationic amino acids (e.g., poly-lysine or poly(L-lysine hydrochloride)).

The coating compound (e.g., polymer) of the instant invention may be linked to at least one targeting ligand. The addition of a targeting ligand permits improved bioavailability. A targeting ligand is a compound that will specifically bind to a specific type of tissue or cell type (e.g., cancerous cell). In a particular embodiment, the targeting ligand is a ligand for a cell surface marker/receptor. The targeting ligand may be an antibody or fragment thereof immunologically specific for a cell surface marker (e.g., protein or carbohydrate) preferentially or exclusively expressed on the targeted tissue or cell type. The targeting ligand may be linked directly to the coating compound or via a linker, particularly to a hydrophilic portion of the amphiphilic compound. Generally, the linker is a chemical moiety comprising a covalent bond or a chain of atoms that covalently attaches the ligand to the coating compound. The linker can be linked to any synthetically feasible position of the ligand and the coating compound. Exemplary linkers may comprise at least one optionally substituted; saturated or unsaturated; linear, branched or cyclic alkyl group or an optionally substituted aryl group (e.g., the linker may comprise from about 1 to about 100 atoms). The linker may also be a polypeptide (e.g., from about 1 to about 10 amino acids, particularly about 1 to about 5). The linker may be non-degradable and may be a covalent bond or any other chemical structure which cannot be substantially cleaved or cleaved at all under physiological environments or conditions.

Notably, all of the coating compounds of a nanoparticle need not be linked to a targeting ligand. Indeed, only a portion of the coating compounds need be linked to a targeting ligand. For example, the ratio of targeting ligand linked to unlinked coating compounds can be 1:1, 1:2, 1:3, 1:4, 1:5, 1:10, or less. Additionally, the nanoparticles of the instant invention may comprise more than one targeting ligand per nanoparticle. The ratio of the different targeting ligands can be controlled by the ratio of components used to synthesize the nanoparticles.

In a particular embodiment, the targeting ligand is a macrophage targeting ligand or a cancer cell targeting ligand. Macrophage targeting ligands include, without limitation, folate receptor ligands (e.g., folate (folic acid) and folate receptor antibodies and fragments thereof (see, e.g., Sudimack et al. (2000) Adv. Drug Del. Rev., 41:147-162)), mannose receptor ligands (e.g., mannose), and formyl peptide receptor (FPR) ligands (e.g., N-formyl-Met-Leu-Phe (fMLF)). For brain targeting and BBB penetration, several physical, chemical, and biological stimuli have been applied (Wong et al. (2011) Adv. Drug Deliv. Rev., 64:686). The mannose receptor is mainly expressed by macrophages and, within the brain, by astrocytes and microglia (Zimmer et al. (2003) Glia 42:89). Folate is a well-established targeting ligand for various cancer and inflammatory diseases. It is also reported that folate derivatives help cross the BBB (Yang et al. (2012) Sub-cellular Biochem., 56:163; Wu et al. (1999) Pharm. Res., 16:415). Mannitol is widely used for endothelial shrinkage and BBB penetration (Doolittle et al. (2000) Cancer 88:637). Except the neuroinflammation itself that causes BBB impairment and leakage, these ligands can maximize the neuroinflammation targeting and BBB penetration of nanoparticles and improve their efficacy in neuroinflammation detection and treatment (Erickson et al. (2012) Neuroimmunomodulation 19:121).

In a particular embodiment, the targeting ligand is a cancer cell targeting ligand. A cancer cell targeting ligand is a compound that will specifically or preferentially bind to a cancer cell rather than any surrounding organ, cell, or tissue. In a particular embodiment, the cancer cell targeting ligand specifically binds a tumor antigen. In a particular embodiment, the targeting ligand is a ligand for the tumor antigen or an antibody or fragment thereof immunologically specific for the tumor antigen. Tumor antigens are well known in the art. Examples of tumor antigens include, without limitation (with an example of an associated cancer in parenthesis): human epithelial growth factor receptor type 2 (Her-2; breast); epithelial cell adhesion molecule (Ep-CAM; breast, colon); prostate stem cell antigen (PSCA; prostate); prostate specific antigen (PSA; prostate); Sigma receptor (sigma-1 receptor, sigma-2 receptor; prostate), CD-44 (prostate; breast), transferrin receptor (prostate, colon); carcinoembryonic antigen (CEA; colon); protein melan-A (also known as melanoma antigen recognized by T-cells 1 (MART-1); melanoma); mesothelin (MSLN; ovarian, mesothelioma); folate receptor (ovarian, breast); and CA-125 (also known as mucin 16; ovarian).

The nanoparticles of the instant invention may be used to deliver any agent(s) or compound(s), particularly bioactive agents (e.g., therapeutic agent or diagnostic/imaging agent) to a cell or a subject (including non-human animals). In a particular embodiment, the encapsulated agent/compound is hydrophobic. In a particular embodiment, the encapsulated agent/compound is hydrophilic. In a particular embodiment, the encapsulated agent/compound is charged (e.g., cationic or anionic). As used herein, the term “bioactive agent” also includes compounds to be screened as potential leads in the development of drugs or plant protecting agents. Bioactive agent include, without limitation, proteins, polypeptides, peptides, glycoproteins, nucleic acids (e.g., DNA, RNA, oligonucleotide, siRNA, antisense, etc.), synthetic and natural drugs, polysaccharides, lipids, peptoides, polyenes, macrocyles, glycosides, terpenes, terpenoids, aliphatic and aromatic compounds, small molecules, and their derivatives and salts. In a particular embodiment, the therapeutic agent is a chemical compound such as a synthetic and natural drug. The nanoparticles of the instant invention may comprise one or more agent or compound. For example, the nanoparticles may comprise more than one therapeutic agent, more than one imaging agent, or one or more therapeutic agents with one or more imaging agent.

The agent/compound (e.g. therapeutic agent) may be hydrophobic, a water insoluble compound, or a poorly water soluble compound. For example, the therapeutic agent may have a solubility of less than about 10 mg/ml, less than 1 mg/ml, more particularly less than about 100 μg/ml, and more particularly less than about 25 μg/ml in water or aqueous media in a pH range of 0-14, particularly between pH 4 and 10, particularly at 20° C. When the agent/compound is hydrophobic, it is preferable that the polymer be an amphiphilic block copolymer, as described hereinabove. In a particular embodiment, when the agent/compound is hydrophobic, the polymer is an amphiphilic block copolymer selected from the group consisting of polyethylene glycol (PEG)-poly(lactic-co-glycolic acid) (PLGA); PEG-polylactic acid (PLA); PEG-polyester; PEG-polycaprolactone (PCL); and PEG-polyanhydride.

The agent/compound (e.g. therapeutic agent), when charged, will typically have a charge (e.g., overall charge) opposite to the ionically charged polymeric segment. For example, the agent/compound (e.g. therapeutic agent) may be positively charged (e.g., protein therapeutics or a small molecule therapeutic). When the agent/compound is positively charged, it is preferable that the polymer be an ionic block copolymer, wherein the ionically charged block is anionic, as described hereinabove. In a particular embodiment, when the agent/compound is positively charged, the polymer is an ionic block copolymer such as PEG-polyglutamic acid or PEG-polyaspartic acid.

The agent/compound (e.g. therapeutic agent) may be negatively charged (e.g., nucleic acid molecules). When the agent/compound is negatively charged, it is preferable that the polymer be an ionic block copolymer, wherein the ionically charged block is cationic, as described hereinabove. In a particular embodiment, when the agent/compound is negatively charged, the polymer is an ionic block copolymer such as PEG-polylysine or PEG-poly(ethyleneimine).

To promote stability, the formed nanoparticles of the instant invention may also be exposed to a cross-linker (i.e., cross-linked). The term “cross-linker” refers to a molecule capable of forming a covalent linkage between compounds (e.g., polymer and therapeutic agent (e.g., protein)). Cross-linkers are well known in the art. In a particular embodiment, the cross-linker is a titrimetric cross-linking reagent. The cross-linker may be a bifunctional, trifunctional, or multifunctional cross-linking reagent. Examples of cross-linkers are provided in, e.g., U.S. Pat. No. 7,332,527. The cross-linker may be cleavable or biodegradable or it may be non-biodegradable or uncleavable under physiological conditions. In a particular embodiment, the cross-linker comprises a bond which may be cleaved in response to chemical stimuli (e.g., a disulfide bond that is degraded in the presence of intracellular glutathione). The cross-linkers may also be sensitive to pH (e.g., low pH). In a particular embodiment, the cross-linker is selected from the group consisting of linkers 3,3′-dithiobis (sulfosuccinimidylpropionate) (DTSSP) and bis(sulfosuccinimidyl)suberate (BS³). 1-Ethyl-3-[3-dimethylaminopropyl]carbodiimide hydrochloride (EDC) and (N-hydroxysulfosuccinimide; Sulfo-NHS) may also be used for cross-linking reactions.

The instant invention encompasses compositions comprising at least one nanoparticle of the instant invention and at least one pharmaceutically acceptable carrier. The compositions of the instant invention may further comprise other agents such as therapeutic agents.

The present invention also encompasses methods for preventing, inhibiting, and/or treating a disease or disorder (e.g., e.g., a neurodegenerative disease, cancer, inflammatory disease, infectious disease, etc.) in a subject. The present invention also encompasses methods for imaging and/or monitoring a disease or disorder (e.g., e.g., a neurodegenerative disease, cancer, inflammatory disease, infectious disease, etc.) in a subject or the efficacy of a therapy. The pharmaceutical compositions of the instant invention can be administered to an animal, in particular a mammal, more particularly a human, in order to treat/inhibit/prevent the disease or disorder. The pharmaceutical compositions and methods of the instant invention may also comprise at least one other bioactive agent, particularly at least one other therapeutic agent. The additional agent may also be administered in separate composition from the nanoparticles of the instant invention. The compositions may be administered at the same time or at different times (e.g., sequentially).

A variety of anti-inflammatory approaches including modulating the activity of various inflammatory mediators such as cytokines and chemokines are considered as possible therapeutic interventions for AD and PD. As the mainstay of treatment for many inflammatory conditions, NSAIDs are widely evaluated for neurodegenerative disorders. Epidemiology studies have shown that long-term use of NSAIDs protects against AD and suppresses its progression. However, large-scale double-blind placebo-controlled clinical trials have not supported the use of NSAIDs in treating neurodegenerative disorders (in t′Veld et al. (2001) N. Engl. J. Med., 345:1515). New therapeutic strategies are critical for neuroinflammation treatment. Mixed-lineage kinases (MLKs) are mitogen-activated protein kinase (MAPK) kinase kinases (MKKKs) that regulate the c-Jun N-terminal kinase (JNK) MAPK signaling cascade and p38 MAPK pathways (Gelbartd et al. (2010) Neurotherpaeutics 7:392). MLK3 (also known as MAP3K11) is the most widely expressed MLK family member. The first generation MLK3 inhibitor, Cephalon (CEP)-1347, has been shown neuroprotection through upregulation of TrkB microglial activation (Pedraza et al. (2009) J. Biol. Chem., 284:32980). MLK3 inhibition by CEP-1347 also showed anti-neuroinflammation by suppressing the production of pro-inflammatory cytokines and chemokines in CNS (Sui et al. (2006) J. Immuno., 177:702).

In a particular embodiment of the instant invention, the nanoparticles comprise at least one therapeutic, i.e., they effect amelioration and/or cure of a disease, disorder, pathology, and/or the symptoms associated therewith. In a particular embodiment, the therapeutic agent is effective for treating, inhibiting, and/or preventing an inflammatory disease or disorder (e.g., neurodegenerative disease and/or neuroinflammation). Inflammatory diseases and disorders include, without limitation, inflammatory bowel disease (IBD), irritable bowel syndrome (IBS), Crohn's disease, rheumatoid arthritis, atherosclerosis, emphysema, chronic obstructive pulmonary disease (COPD), ulcerative colitis, multiple sclerosis, and neurodegenerative disease and/or neuroinflammation diseases. Neurodegenerative diseases or disorders include without limitation: Alzheimer's disease, Huntington's disease, Parkinson's disease, Lewy Body disease, amyotrophic lateral sclerosis, and prion disease. In a particular embodiment, the therapeutic agent is an anti-inflammatory including, without limitation: cytokine, chemokine, kinase inhibitor (e.g., MLK inhibitor or MLK3 inhibitor), non-steroidal anti-inflammatory drug (NSAID; e.g., salicylates (e.g., aspirin (acetylsalicylic acid), diflunisal, salsalate), propionic acid derivatives (e.g., ibuprofen, dexibuprofen, naproxen, etc.), acetic acid derivatives (e.g., indomethacin, sulindac, etc.), enolic acid (oxicam) derivatives (e.g., piroxicam, meloxicam, etc.), fenamates (e.g., mefenamic acid, meclofenamic acid, etc.), COX-2 inhibitors (e.g., celecoxib)), nuclear factor-κB (NF-κB) inhibitor, superoxide dismutase, or catalase. In a particular embodiment, the therapeutic agent is a MLK3 inhibitor.

In a particular embodiment, the therapeutic agent is effective for treating, inhibiting, and/or preventing cancer (e.g., a chemotherapeutic agent). In a particular embodiment, the cancer may be selected from the group consisting of, without limitation, cancers of the prostate, colorectum, pancreas, cervix, stomach, endometrium, brain, liver, bladder, ovary, testis, head, neck, skin, melanoma, basal carcinoma, mesothelial lining, white blood cells, lymphoma, leukemia, esophagus, breast, muscle, connective tissue, lung, small-cell lung carcinoma, non-small-cell carcinoma, adrenal gland, thyroid, kidney, or bone; glioblastoma, mesothelioma, renal cell carcinoma, gastric carcinoma, sarcoma, choriocarcinoma, cutaneous basocellular carcinoma, and testicular seminoma.

In a particular embodiment, the therapeutic agent is effective for treating, inhibiting, and/or preventing an infectious disease or disorder. In a particular embodiment, the therapeutic agent is an antimicrobial agent to treat, inhibit, and/or prevent a microbial infection (e.g., a bacterial, viral infection).

### IV. ADMINISTRATION

The instant invention encompasses compositions comprising at least one nanoparticle of the instant invention and, optionally, at least one pharmaceutically acceptable carrier. As stated hereinabove, the nanoparticle may comprise more than one encapsulated compound (e.g., therapeutic agent and/or imaging agent). In a particular embodiment, the composition comprises a first nanoparticle comprising a first encapsulated compound(s) and a second nanoparticle comprising a second encapsulated compound(s), wherein the first and second encapsulated compounds are different. The compositions of the instant invention may further comprise other therapeutic agents.

The present invention also encompasses methods for preventing, inhibiting, and/or treating a disease or disorder (e.g., a neurodegenerative disease, cancer, inflammatory disease, infectious disease, etc.). The pharmaceutical compositions of the instant invention can be administered to an animal, in particular a mammal, more particularly a human, in order to treat/inhibit the disease/disorder. The pharmaceutical compositions of the instant invention may also comprise at least one other therapeutic agent. The additional therapeutic agent may also be administered in a separate composition from the nanoparticles of the instant invention. The compositions may be administered at the same time or at different times (e.g., sequentially).

As explained hereinabove, the instant invention also encompasses methods of monitoring biodistribution of the encapsulated compound (e.g., therapeutic agent). In a particular embodiment, the method comprises administering the nanoparticles of the invention to a subject and performing at least one MRI procedure, thereby determining the location of the nanoparticles and the encapsulated compounds. The methods may comprise performing more than one MRI procedure at different times. The methods may further comprise assaying for additional imaging agents, if present. The monitoring of the distribution of the encapsulated compound allows for real time assessment of the therapy (e.g., for personalized medicine) and allow for the optimization of the treatment to direct more of the encapsulated compound to the desired target and reduce toxicity. For example, the route of administration, frequency of administration, amount of dose, and/or targeting of the nanoparticle may be modified.

The dosage ranges for the administration of the compositions of the invention are those large enough to produce the desired effect (e.g., curing, relieving, treating, and/or preventing the disease or disorder, the symptoms of it, or the predisposition towards it). The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The nanoparticles described herein will generally be administered to a patient as a pharmaceutical preparation. The term “patient” as used herein refers to human or animal subjects. These nanoparticles may be employed therapeutically, under the guidance of a physician. While the therapeutic agents are exemplified herein, any bioactive agent may be administered to a patient, e.g., a diagnostic or imaging agent.

The compositions comprising the nanoparticles of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the nanoparticles in the chosen medium may be varied and the medium may be chosen based on the desired route of administration of the pharmaceutical preparation. Except insofar as any conventional media or agent is incompatible with the nanoparticles to be administered, its use in the pharmaceutical preparation is contemplated.

The dose and dosage regimen of nanoparticles according to the invention that are suitable for administration to a particular patient may be determined by a physician considering the patient's age, sex, weight, general medical condition, and the specific condition for which the nanoparticles are being administered and the severity thereof. The physician may also take into account the route of administration, the pharmaceutical carrier, and the nanoparticle's biological activity.

Selection of a suitable pharmaceutical preparation will also depend upon the mode of administration chosen. For example, the nanoparticles of the invention may be administered by direct injection or intravenously. In this instance, a pharmaceutical preparation comprises the nanoparticle dispersed in a medium that is compatible with the site of injection.

Nanoparticles of the instant invention may be administered by any method. For example, the nanoparticles of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In a particular embodiment, the nanoparticles are administered intravenously or intraperitoneally. Pharmaceutical preparations for injection are known in the art. If injection is selected as a method for administering the nanoparticle, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a nanoparticle of the present invention as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, oral, direct injection, intracranial, and intravitreal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of nanoparticles may be determined by evaluating the toxicity of the molecules or cells in animal models. Various concentrations of nanoparticles in pharmaceutical preparations may be administered to mice, and the minimal and maximal dosages may be determined based on the beneficial results and side effects observed as a result of the treatment. Appropriate dosage unit may also be determined by assessing the efficacy of the nanoparticle treatment in combination with other standard drugs. The dosage units of nanoparticle may be determined individually or in combination with each treatment according to the effect detected.

The pharmaceutical preparation comprising the nanoparticles may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The instant invention encompasses methods of treating a disease/disorder comprising administering to a subject in need thereof a composition comprising a nanoparticle of the instant invention and, particularly, at least one pharmaceutically acceptable carrier. Nanoparticles of the instant invention can be injected directly to a subject or through injection with macrophages that have internalized nanoparticles ex vivo/in vitro. In a particular embodiment of the instant invention, the instant methods comprise treating the subject via an ex vivo therapy. In particular, the method comprises removing cells from the subject, exposing/contacting the cells in vitro to the nanoparticles of the instant invention, and returning the cells to the subject. In a particular embodiment, the cells comprise macrophage. Other methods of treating the disease or disorder may be combined with the methods of the instant invention may be co-administered with the compositions of the instant invention.

The instant also encompasses delivering the nanoparticle of the instant invention to a cell in vitro (e.g., in culture). The nanoparticle may be delivered to the cell in at least one carrier.

### V. DEFINITIONS

The following definitions are provided to facilitate an understanding of the present invention:

The singular forms “a,” “an,” and “the” include plural referents unless the context clearly dictates otherwise.

As used herein, the term “subject” refers to an animal, particularly a mammal, particularly a human.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

As used herein, the term “lipophilic” refers to the ability to dissolve in lipids.

As used herein, the term “hydrophilic” means the ability to dissolve in water.

As used herein, the term “amphiphilic” means the ability to dissolve in both water and lipids. Typically, an amphiphilic compound comprises a hydrophilic portion and a lipophilic portion.

The term “substantially cleaved” may refer to the cleavage of the amphiphilic polymer from the protein of the conjugates of the instant invention, preferably at the linker moiety. “Substantial cleavage” occurs when at least 50% of the conjugates are cleaved, preferably at least 75% of the conjugates are cleaved, more preferably at least 90% of the conjugates are cleaved, and most preferably at least 95% of the conjugates are cleaved.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween® 80, Polysorbate 80), emulsifier, buffer (e.g., Tris HCl, acetate, phosphate), bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “biodegradable” or “biodegradation” is defined as the conversion of materials into less complex intermediates or end products by solubilization hydrolysis under physiological conditions, or by the action of biologically formed entities which can be enzymes or other products of the organism. The term “non-degradable” refers to a chemical structure that cannot be cleaved under physiological conditions.

The term “alkyl,” as employed herein, includes both straight and branched chain hydrocarbons containing about 1 to about 20 carbons, particularly about 1 to about 15, particularly about 5 to about 15 carbons in the main chain. The hydrocarbon chain of the alkyl groups may be interrupted with heteroatoms such as oxygen, nitrogen, or sulfur atoms. Each alkyl group may optionally be substituted with substituents which include, for example, alkyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol.

The term “aryl,” as employed herein, refers to monocyclic and bicyclic aromatic groups containing 6 to 10 carbons in the ring portion. Aryl groups may be optionally substituted through available carbon atoms. The aromatic ring system may include heteroatoms such as sulfur, oxygen, or nitrogen.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “treat” as used herein refers to any type of treatment that imparts a benefit to a patient afflicted with a disease or disorder, including improvement in the condition of the patient (e.g., in one or more symptoms), delay in the progression of the condition, etc.

As used herein, the term “prevent” refers to the prophylactic treatment of a subject who is at risk of developing a condition resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to modulate neuroinflammation in a subject.

The term “tumor antigen” refers to an antigen associated with certain tumor. Typically, tumor antigens are found in significant amounts in tumors, but are found in lower amounts or not at all in normal tissues.

The term “antimicrobials” as used herein indicates a substance that kills or inhibits the growth of microorganisms such as bacteria, fungi, viruses, or protozoans.

As used herein the term “antibiotic” refers to a molecule that inhibits bacterial growth or pathogenesis. Antibiotics include, without limitation, β-lactams (e.g., penicillins and cephalosporins), vancomycins, bacitracins, macrolides (e.g., erythromycins, clarithromycin, azithromycin), lincosamides (e.g., clindomycin), chloramphenicols, tetracyclines (e g, immunocycline, chlortetracycline, oxytetracycline, demeclocycline, methacycline, doxycycline and minocycline), aminoglycosides (e.g., gentamicins, amikacins, neomycins, amikacin, streptomycin, kanamycin), amphotericins, cefazolins, clindamycins, mupirocins, sulfonamides and trimethoprim, rifampicins, metronidazoles, quinolones, fluoroquinolones (e.g., ciprofloxacin, levofloxacin, moxifloxacin), novobiocins, polymixins, gramicidins, vancomycin, imipenem, meropenem, cefoperazone, cefepime, penicillin, nafcillin, linezolid, aztreonam, piperacillin, tazobactam, ampicillin, sulbactam, clindamycin, metronidazole, levofloxacin, a carbapenem, linezolid, rifamycins (e.g., rifampin, rifabutin), clofazimine, and metronidazole.

As used herein, the term “antiviral” refers to a substance that destroys a virus or suppresses replication (reproduction) of the virus.

An “antibody” or “antibody molecule” is any immunoglobulin, including antibodies and fragments thereof (e.g., scFv), that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, a “ligand” refers to a biomolecule, such as a protein or polypeptide, which specifically and/or selectively binds another polypeptide or protein. In a particular embodiment, the term “ligand” refers to a biomolecule which binds to a specific receptor protein located on the surface of a cell.

Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, Pseudomonas exotoxin, and others listed above; thereby generating an immunotoxin when conjugated or fused to an antibody); alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

As used herein, an “inflammatory disease” refers to a disease caused by or resulting from or resulting in inflammation. The term “inflammatory disease” may also refer to a dysregulated inflammatory reaction that causes an exaggerated response by macrophages, granulocytes, and/or T-lymphocytes leading to abnormal tissue damage and cell death.

The following example provides illustrative methods of practicing the instant invention, and is not intended to limit the scope of the invention in any way.

### EXAMPLE

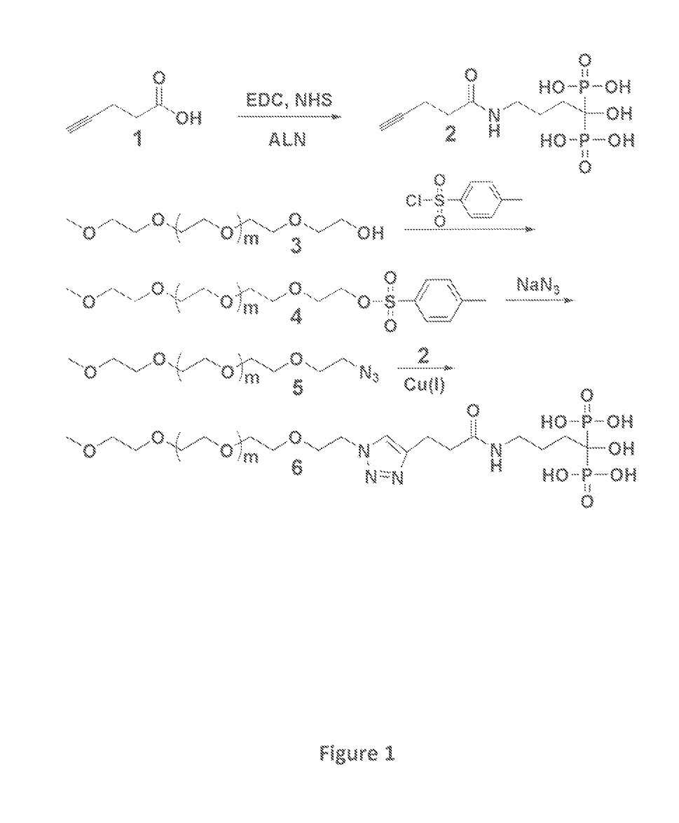

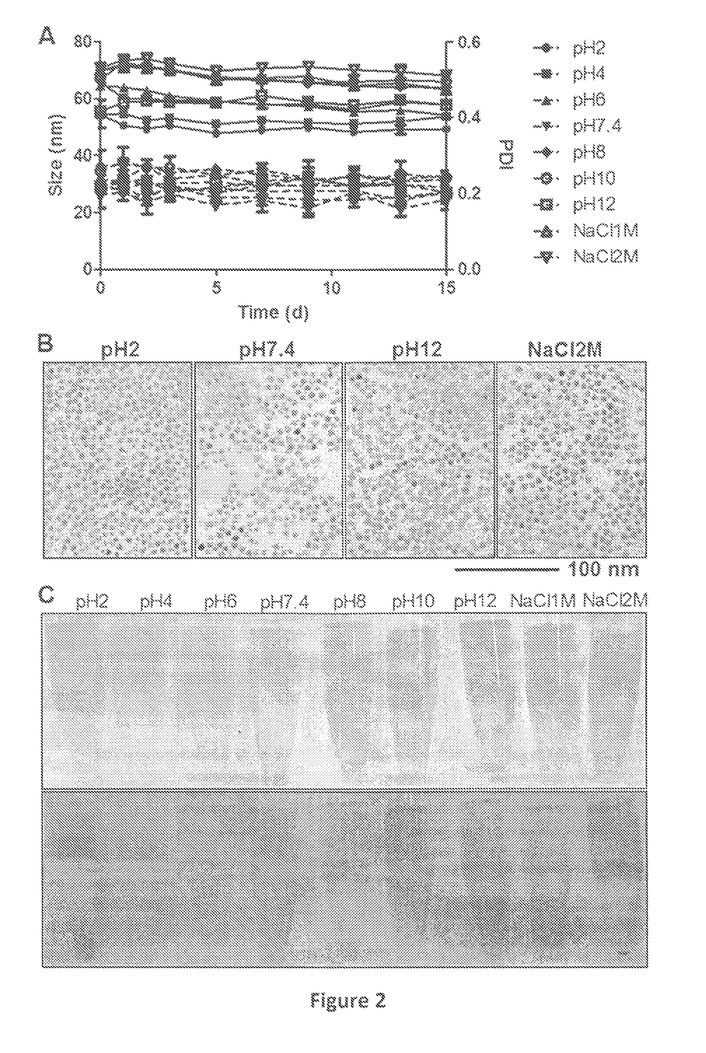

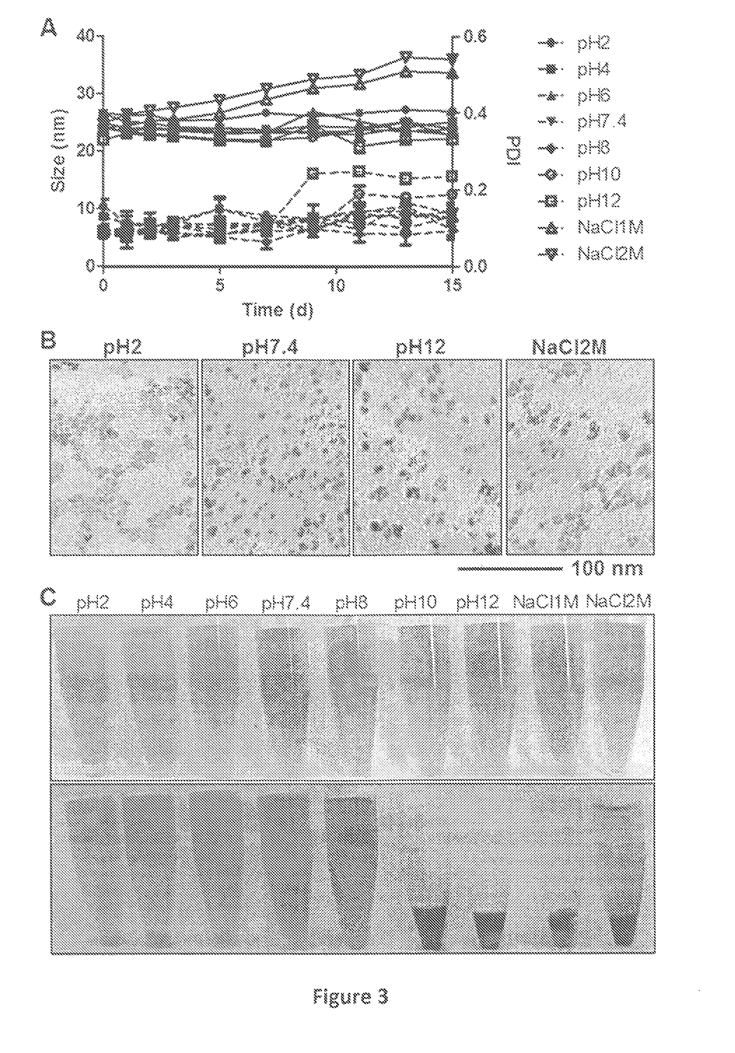

It is shown herein that bisphosphonates and derivatives thereof are ideal agents for stable SPIO coating. FIG. 1 provides a schematic of a synthesis protocol for alendronate conjugates to polyethylene oxide (ALN-PEO). ALN-PEO coating of USPIOs occurs within minutes (e.g., by mixing (e.g., at room temperature) and removal of free components (e.g., by centrifugation or dialyzation)), thereby demonstrating its outstanding coating ability. An in vitro evaluation also showed that this coating is very stable in 0-12 pH buffer solutions and 0-2 M salt solutions (FIG. 2). No significant size increase and polydispersity index (PDI) change were found after a 2-week storage (FIG. 2A). TEM studies also showed that no USPIO aggregates formed (about 10 nm) in all of the tested conditions after a 1-month storage (FIG. 2B). In contrast, ferumoxytol showed a minimal size increase in high salt solutions (1-2 M) and a minimal PDI shift in high pH buffers (pH 10-12), and particle aggregation was found with longer storage (FIG. 3). Ferumoxytol is a superparamagnetic iron oxide nanoparticle that has a polyglucose carboxy-methylether coating.

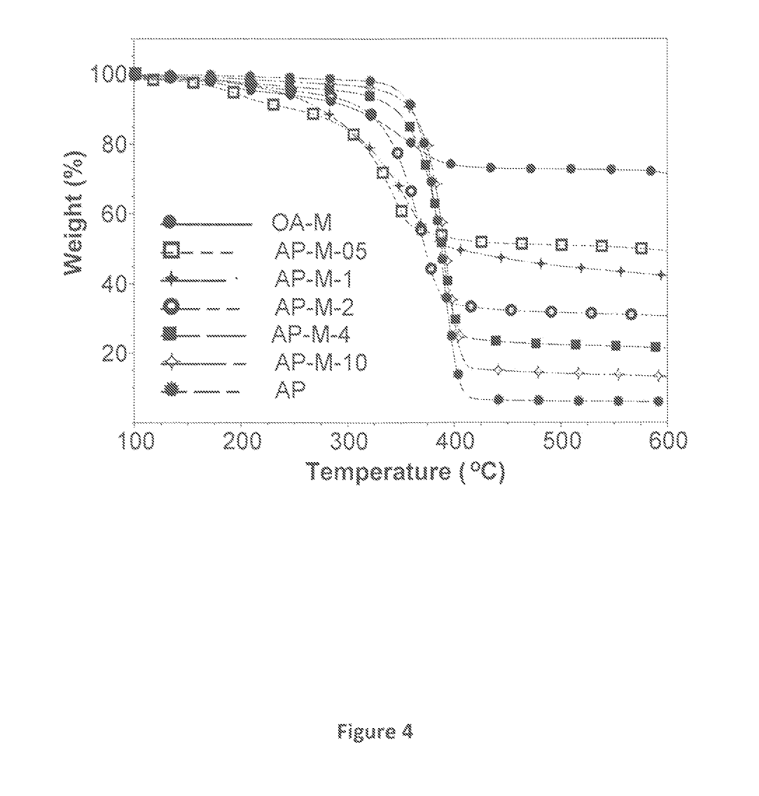

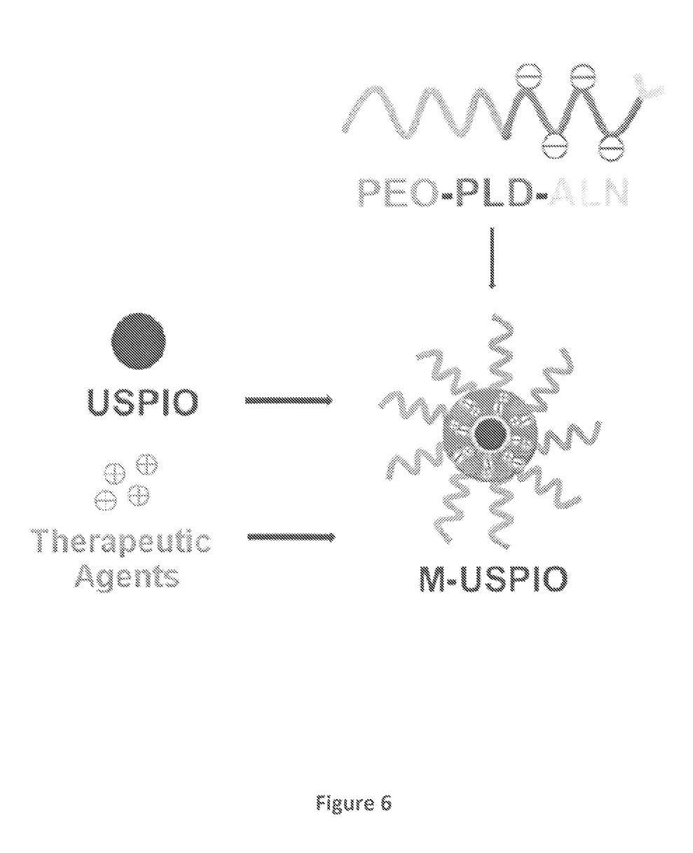

For drug delivery, the core of a block ionomer complex (BIC) is formed between the ionic chain blocks of hydrophilic block copolymers and the oppositely charged pharmaceutical agents for encapsulation. The nonionic chain blocks of hydrophilic block copolymers such as poly(ethylene oxide) (PEO) serve as corona to prevent the aggregation and macroscopic phase separation of BIC (Oh et al. (2006) J. Controlled Rel., 115:9; Kabanov et al. (1998) Adv. Drug Del. Rev., 30:49). This unique core-corona nano-carrier structure has been widely used for the delivery of protein, gene, nucleic acids, and small ionic molecules (Kabanov et al. (1998) Adv. Drug Del. Rev., 30:49; Zhao et al. (2011) Nanomedicine 6:25; Oberoi et al. (2011) J. Controlled Rel., 153:64; Kim et al. (2009) Polymer Sci. A, 51:708). BIC is relatively stable even with the polyion chain completely neutralized, but BIC display transitions induced by changes in pH, salt concentration, and temperature. To prohibit its premature disintegration upon systemic administration, chemical cross-linking of ionic chain blocks are widely used to stabilize BIC core, which may decrease or destroy the activity of protein therapeutics (Zhao et al. (2011) Nanomedicine 6:25; Oberoi et al. (2011) J. Controlled Rel., 153:64; Kim et al. (2009) Polymer Sci. A, 51:708). To address this potential problem, the bisphosphonate-modified polymer ALN-PEO is used to strongly coat onto USPIOs serving as stable core anchor. As seen in FIG. 4, ALN-PEO can strongly coat USPIOs with a well-tunable ALN-PEO to USPIO weight ratio of about 2-8.5. This ratio can be further optimized if high molecular weight block copolymer is applied. Thermogravimetric analysis (TGA) results showed that the coating is very efficient with almost 100% of the polymer coated onto USPIOs within 30 minutes (FIG. 4). This coating can be used for the generation of USPIO anchored stable BIC nanoparticles (e.g., nano-theranostics; FIG. 6). This tunable coating provides significant flexibility to regulate drug-loading capacity and incorporate fixed ratios of multimodel targeting ligands into a single USPIO particle in a programmed and reproducible manner. Modified USPIOs may serve as MRI imaging agents (e.g., for neuroinflammation detection), carriers of therapeutic agents (e.g., MLK3 inhibitors), and/or form stable BICs with charged polymers (e.g., negatively charged polymers such as PLD-PEO).

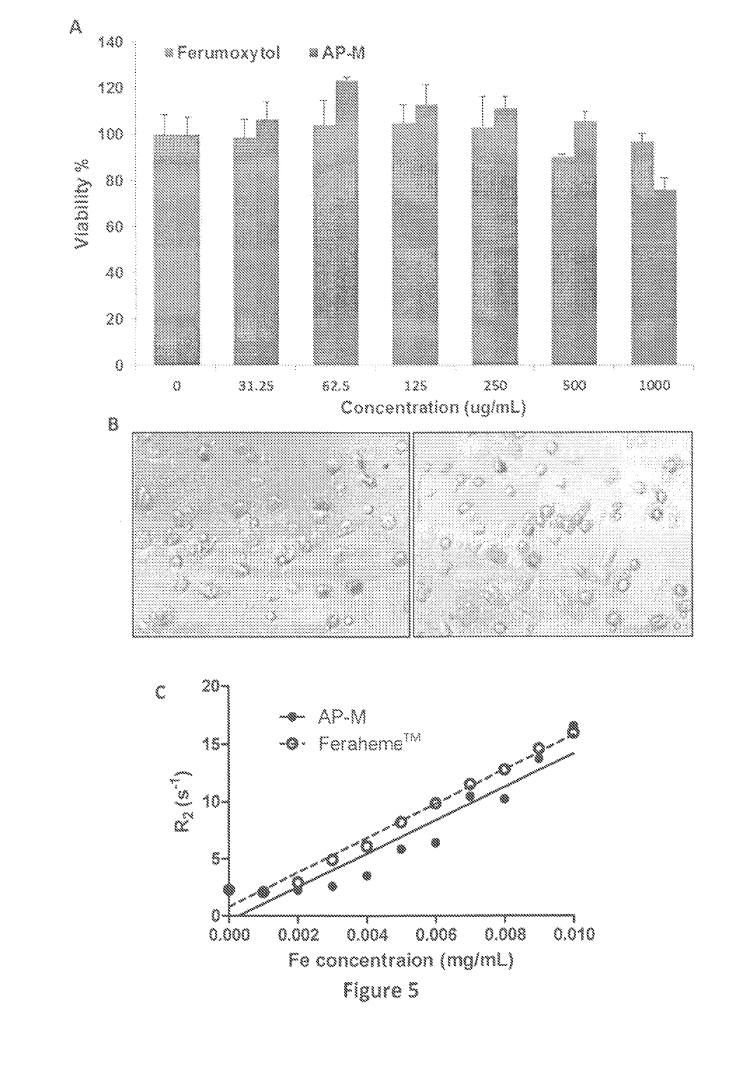

MRI relaxivity studies showed that ALN-PEO coated USPIOs have the similar T2-weighted relaxivity constant to that of the CNS MRI contrast agent ferumoxytol (1459.4 versus 1502.8 s⁻¹ mlmg⁻¹; FIG. 5C). FIG. 5 also shows toxicity and uptake of ALN-PEO coated USPIOs compared to ferumoxytol. As a benefit from their high coating efficiency, ALN-polymers can be firstly functionalized with different ligands and then coated onto USPIOs with a programmable and reproducible manner.

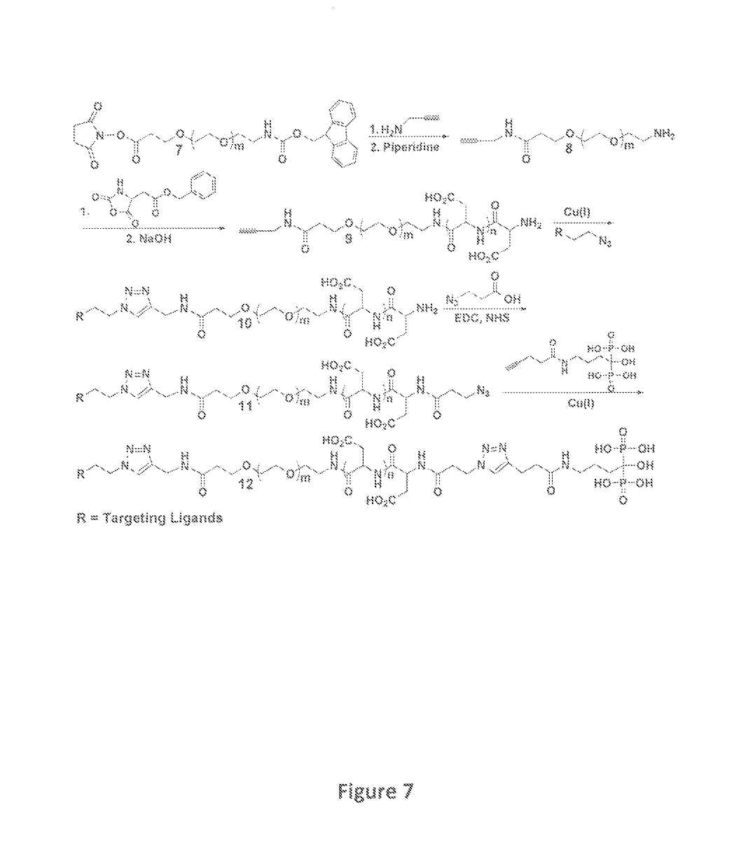

FIG. 7 provides a schematic for the synthesis of L-PEO-PLD-ALN. Briefly, acetylene-functionalized PEO-PLD was synthesized by N-caboxyanhydride (NCA) polymerization. Optional targeting ligands (e.g., mannose, folate, or mannitol) may be functionalized with an azido-group and then conjugated on acetylene-functionalized PEO-PLO via the versatile click chemistry (Kolb et al. (2001) Angew Chem. Int. Ed. Engl. 40:2004). Finally, the PLD block termini of L-PEO-PLDs are functionalized with an azido-group and clicked with acetylene-ALN to synthesize the desired ligand-PEO-PLD-ALN polymers with near quantitative yield. The coating of SPIO with ALN-PEO-PLD (specifically, ALN-PEO₁₁₀-PLD₂₀ (SALN)) proved to be stable as seen in Table 1. The chemical structure of the polymer is:

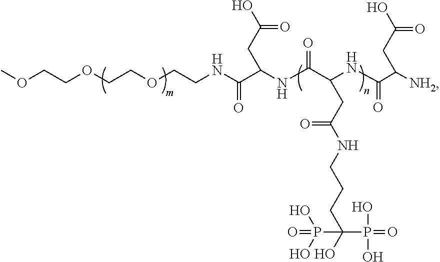

wherein m is 110 and n is 20. The polymers are then dissolved in water and then mixed with USPIOs in water for coating. After one hour, the mixture is dialyzed under 25 kDa MWCO dialysis tubing in order to remove uncoated polymer.

TABLE 1
Nanoparticle diameter (nm) and PDI
after the indicated number of days.

| Time (days) | Size | PDI |
|---|---|---|
| 0 | 82.53 | 0.174 |
| 1 | 86.98 | 0.225 |
| 3 | 81.29 | 0.191 |
| 5 | 78.98 | 0.187 |
| 7 | 91.41 | 0.264 |
| 11 | 95.10 | 0.267 |
| 17 | 88.36 | 0.245 |

As stated hereinabove, nanoparticle formation proceeds rapidly. For example, for the encapsulation of hydrophobic drugs into the nanoparticle system, PEO-PLGA-ALN and oleic acid coated USPIOs were dissolved into tetrahydrofuran (THF). After 2 hours stirring at room temperature, hydrophobic drugs were then added and dissolved into the mixture. The mixture was then added into water or buffer drop-by-drop with stirring and the THF was then evaporated under vacuum. The mixture was finally centrifuged to remove free drugs at 1000 g for 10 minutes. The supernatant was collected as pure drug loaded USPIO nanoparticles.

As an example of the encapsulation of hydrophilic ionic therapeutics into the nanoparticle system, PEO-PolyAsp-ALN (for cationic therapeutics) or PEO-PolyLysine-ALN (for anionic therapeutics) and hydrophilic USPIOs were dissolved into water. After 2 hours stirring at room temperature, ionic therapeutics were added and dissolved into the mixture. The mixture was then centrifuged to remove free drug at 15000 g for 30 minutes. The pellets were collected as pure drug loaded USPIO nanoparticles.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A nanoparticle comprising a metal nanoparticle core and a polymer linked to a metal binding moiety, wherein said polymer is bound to the metal nanoparticle by the metal binding moiety and wherein said polymer coats said metal nanoparticle core.

2. The nanoparticle of claim 1, wherein said polymer is a hydrophilic or an amphiphilic block copolymer.

3. The nanoparticle of claim 2, wherein at least one hydrophilic block of said amphiphilic block copolymer comprises polyethylene oxide.

4. The nanoparticle of claim 2, wherein at least one hydrophobic block of said amphiphilic block copolymer comprises a polyester or polyanhydride.

5. The nanoparticle of claim 1, wherein said polymer is an ionic block copolymer, wherein said ionic block copolymer comprises at least one ionic block and at least one hydrophilic block.

6. The nanoparticle of claim 5, wherein said at least one ionic block comprises a poly(amino acid).

7. The nanoparticle of claim 5, wherein said at least one hydrophilic block comprises polyethylene oxide.

8. The nanoparticle of claim 1, wherein said nanoparticle further comprises a therapeutic agent.

9. The nanoparticle of claim 8, wherein said therapeutic agent is an anti-inflammatory.

10. The nanoparticle of claim 8, wherein said therapeutic agent is a chemotherapeutic agent.

11. The nanoparticle of claim 8, wherein said therapeutic agent is an antimicrobial.

12. The nanoparticle of claim 9, wherein said therapeutic agent is a mixed lineage kinase 3 (MLK3) inhibitor or nonsteroidal anti-inflammatory drug.

13. The nanoparticle of claim 8, wherein said therapeutic agent is hydrophobic and said polymer is an amphiphilic block copolymer.

14. The nanoparticle of claim 8, wherein said therapeutic agent is charged and said polymer is an ionic block copolymer and wherein the charge of the ionic block copolymer is the opposite of the therapeutic agent.

15. The nanoparticle of claim 1, wherein said polymer is linked to at least one targeting ligand.

16. The nanoparticle of claim 15, wherein said targeting ligand is a macrophage targeting ligand or a cancer cell targeting ligand.

17. The nanoparticle of claim 1, wherein said metal nanoparticle is paramagnetic or a quantum dot.

18. The nanoparticle of claim 17, wherein said metal nanoparticle is an ultrasmall superparamagnetic iron oxide (USPIO) particle.

19. The nanoparticle of claim 1, wherein said metal binding moiety comprises bisphosphonate, pyrophosphate, or a derivative thereof.

20. A composition comprising at least one nanoparticle of claim 1 and at least one pharmaceutically acceptable carrier.

21. A method for treating or inhibiting a neurodegenerative disease in a subject in need thereof, said method comprising administering to said subject at least one nanoparticle of claim 1, wherein said nanoparticle further comprises an anti-inflammatory.

22. The method of claim 21, wherein said neurodegenerative disease is Alzheimer's disease or Parkinson's disease.

23. A method for treating or inhibiting cancer in a subject in need thereof, said method comprising administering to said subject at least one nanoparticle of claim 1, wherein said nanoparticle further comprises a chemotherapeutic agent.

24. A method for monitoring the biodistribution of a therapeutic agent in a subject, said method comprising:
a) administering to said subject at least one composition of claim 18, wherein said nanoparticle further comprises a therapeutic agent; and
b) performing at least one magnetic resonance imaging or optical imaging procedure, thereby determining the distribution of the therapeutic agent within the subject.
